(19) United States
(12) Patent Application Publication
(10) Pub. No.: US 2024/0148012 A1
(43) Pub. Date: May 9, 2024

(54) SYSTEMS AND METHODS OF PRODUCING FAT TISSUE FOR CELL-BASED MEAT PRODUCTS

(71) Applicant: Terasaki Institute for Biomedical Innovation, Los Angeles, CA (US)

(72) Inventors: Seyedvahid Hosseini, Newbury Park, CA (US); Alireza Khademhosseini, Los Angeles, CA (US)

(73) Assignee: Terasaki Institute for Biomedical Innovation, Los Angeles, CA (US)

(21) Appl. No.: 18/281,000

(22) PCT Filed: Mar. 9, 2022
(86) PCT No.: PCT/US2022/019609

§ 371 Date: Sep. 8, 2023

Related U.S. Application Data
(60) Provisional application No. 63/314,171, filed on Feb. 25, 2022.
(60) Provisional application No. 63/314,191, filed on Feb. 25, 2022.
(60) Provisional application No. 63/300,577, filed on Jan. 18, 2022.
(60) Provisional application No. 63/279,617, filed on Nov. 15, 2021.
(60) Provisional application No. 63/279,631, filed on Nov. 15, 2021.
(60) Provisional application No. 63/279,642, filed on Nov. 15, 2021.
(60) Provisional application No. 63/279,644, filed on Nov. 15, 2021.
(60) Provisional application No. 63/164,397, filed on Mar. 22, 2021.
(60) Provisional application No. 63/164,387, filed on Mar. 22, 2021.
(60) Provisional application No. 63/159,403, filed on Mar. 10, 2021.

(51) Int. Cl. A23D 7/005; A23J 3/22

(52) U.S. Cl. CPC A23D 7/0053; A23J 3/227

(57) ABSTRACT

The present disclosure generally relates, in certain aspects, to cultivated meat and other cultivated animal-derived products. In some embodiments, such products may include fat replicas, which may improve taste, appearance, etc. In some cases, a fat replica can be formed by forming an emulsion of fat and a non-human blood plasma, then causing the blood plasma to crosslink and/or clot, e.g., forming a hydrogel containing the fat emulsion. In some cases, the fat replica may be used to make a cultivated meat product, e.g., by combining with muscle replicas, lysate of non-human red blood cells, etc. Other embodiments are generally directed to methods of making or using such fat replicas, the microcarriers, or the cultivated meat products, kits involving these, or the like.

### RELATED APPLICATIONS

This application claims the benefit of U.S. Provisional Patent Application Ser. No. 63/159,403, filed Mar. 10, 2021, entitled “Constructs for Meat Cultivation and Other Applications”; U.S. Provisional Patent Application Ser. No. 63/279,617, filed Nov. 15, 2021, entitled “Constructs Comprising Fibrin or Other Blood Products for Meat Cultivation and Other Applications”; U.S. Provisional Patent Application Ser. No. 63/279,631, filed Nov. 15, 2021, entitled, “Methods and Systems of Preparing Cultivated Meat from Blood or Cellular Biomass”; U.S. Provisional Patent Application Ser. No. 63/279,642, filed Nov. 15, 2021, entitled, “Systems and Methods of Producing Fat Tissue for Cell-Based Meat Products”; U.S. Provisional Patent Application Ser. No. 63/279,644, filed Nov. 15, 2021, entitled “Production of Heme for Cell-Based Meat Products”; US Provisional Patent Application Serial No. U.S. 63/300,577, filed Jan. 18, 2022, entitled “Animal-Derived Antimicrobial Systems and Methods”; U.S. Provisional Patent Application Ser. No. 63/164,397, filed Mar. 22, 2021, entitled “Growth Factor for Laboratory Grown Meat”; U.S. Provisional Patent Application Ser. No. 63/164,387, filed Mar. 22, 2021, entitled, “Methods of Producing Animal Derived Products”; U.S. Provisional Patent Application Ser. No. 63/314,171, filed Feb. 25, 2022, entitled “Growth Factors for Laboratory Grown Meat and Other Applications”; and U.S. Provisional Patent Application Ser. No. 63/314,191, filed Feb. 25, 2022, entitled “Methods and Systems of Producing Products Such as Animal Derived Products.” Each of these is incorporated herein by reference in its entirety.

### FIELD

The present disclosure generally relates to cultivated meat and other cultivated animal-derived products.

### BACKGROUND

Cultivated meat, or cell-based meat, is meat that is produced using in vitro cell culture or bioreactors, instead of being harvested from live animals. In many cases, the meat that is produced may include muscle cells and fat cells. Such meat may include, for example, chicken, beef, pork, or fish. Such technologies have the potential to revolutionize agriculture, for example, by decreasing the amount of land necessary to produce meat, avoiding unethical farming of animals, or increasing the available food supply. However, it is still difficult and expensive to integrate fat into products such as cultivated meat products, and thus improvements are needed.

### SUMMARY

The present disclosure generally relates to cultivated meat and other cultivated animal-derived products. The subject matter of the present disclosure involves, in some cases, interrelated products, alternative solutions to a particular problem, and/or a plurality of different uses of one or more systems and/or compositions.

Certain embodiments of the present disclosure are generally directed to fat replicas that can be added to cultivated meat products, or other applications such as discussed herein, to mimic the fat profile (e.g., the taste and/or texture) of the products. Some embodiments, for example, are directed to compositions comprising a fat replica. For example, in some cases, the fat replica may replicate certain desirable fat contents, textures, tastes, mechanical properties, rheological properties (e.g., elastic modulus, loss modulus, etc.), or the like. For example, in one set of embodiments, a fat replica comprises a microcarrier or scaffold, and non-human fat cells, such as adipose cells. In some embodiments, the fat replica may comprise a fat emulsion, a non-human blood plasma, and a hydrogel.

In some embodiments, the fat may comprise animal fat and/or plant fat. Plant-based fats can include any fat product obtained or extracted from a plant, e.g., oleic acid, canola oil, or others such as those described herein. In one set of embodiments, the fat replica comprises a solution that includes a crosslinked hydrogel.

In some embodiments, the cultivated meat may be grown in a bioreactor comprising a cell culture media that is, at least partially, comprised of blood products. The blood products may be harvested from a human or non-human and may comprise components such as platelet rich plasma (PRP), platelet poor plasma (referred to as plasma), a platelet concentrate, a lysate of red blood cells, a platelet lysate (PL), growth factors, proteins, cytokines, or the like. In another embodiment, the non-human blood plasma may be used as a nutrient source in a bioreactor. In certain embodiments, the serum is fetal bovine serum. In some cases, the blood may be obtained from commercial vendors. However, in some embodiments, the non-human blood plasma may be obtained from living animal donors.

In addition, certain embodiments as discussed herein are directed toward methods of producing fat replicas. For example, in some cases, fat may be dispersed and/or encapsulated within a hydrogel. For example, in one set of embodiments, fat may be dispersed in a solution comprising non-human blood plasma. Plasma contains multiple compounds capable of stabilizing a colloidal fat dispersion, for example, bile acids, albumin, and lipids. The plasma may be caused to clot, which may stabilize and/or entrap fat, for example, as an emulsion. In some embodiments, the fat may be dispersed using a surfactant in a solution comprising a polymer. In some embodiments the polymer may be caused to gel, thus entrapping the fat emulsion. In addition, in some embodiments, a fat replica may include cultured fat cells, such as adipose cells. For instance, the fat cells may be cultured on a microcarrier, and/or within a bioreactor, to generate a fat replica.

One aspect is generally directed to a fat replica. In one set of embodiments, the fat replica comprises a fat emulsion, and a hydrogel comprising crosslinked non-human blood plasma.

Another aspect is generally directed to a cultivated meat product. According to one set of embodiments, the cultivated meat product comprises a tissue mass of at least 10 g, comprising a fat emulsion and a crosslinked non-human blood plasma. In some cases, the tissue mass comprises no more than 0.1 wt % saturated fat.

In another set of embodiments, the cultivated meat product comprises a tissue mass of at least 10 g, comprising fat and non-human blood plasma. In some embodiments, the tissue mass has no more than 0.1 wt % cholesterol.

The cultivated meat product, in accordance with yet another set of embodiments, comprises a tissue mass of at least 10 g, comprising an emulsion of fat and non-human blood plasma. In certain cases, the tissue mass is substantially free of cholesterol.

Yet another aspect is generally directed to a method. In one set of embodiments, the method is a method of forming a fat replica. In certain embodiments, the method comprises mixing fat and a non-human blood plasma to form an emulsion, and causing the plasma within the emulsion to clot.

In another set of embodiments, the method comprises mixing fat, a hydrogel, and a surfactant to form an emulsion, crosslinking the hydrogel within the emulsion, and mixing the crosslinked hydrogel and non-human animal cells to produce a tissue mass of at least 10 g.

The method, in still another set of embodiments, comprises mixing fat and a non-human blood plasma, and forming a stabilized structure by causing the plasma to clot.

In yet another set of embodiments, the method comprises culturing non-human fat cells on microcarriers comprising fibrin, mixing the cultured cells with a hydrogel, and crosslinking the hydrogel.

In another set of embodiments, the method comprises culturing non-human fat cells on microcarriers comprising fibrin, mixing the cultured cells with non-human blood plasma, and causing the plasma to clot.

Still another set of embodiments is directed to a method comprising mixing fat and a non-human blood plasma, and crosslinking the plasma.

In another aspect, the present disclosure encompasses methods of making one or more of the embodiments described herein, for example, cultivated meat. In still another aspect, the present disclosure encompasses methods of using one or more of the embodiments described herein, for example, cultivated meat.

Other advantages and novel features of the present disclosure will become apparent from the following detailed description of various non-limiting embodiments of the disclosure when considered in conjunction with the accompanying figures.

### BRIEF DESCRIPTION OF THE DRAWINGS

Non-limiting embodiments of the present disclosure will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1 illustrates a process of forming a fat replica using fibrin hydrogels in accordance with one embodiment;

FIG. 2 illustrates an optical image of a fat replica following staining with Oil-O′ Red to highlight the fat component of a tissue construct, in another embodiment;

FIG. 3 illustrates a chylomicron, in accordance with yet another embodiment;

FIG. 4A illustrates a photomicrograph of a fat replica comprising alginate and croconate oil in accordance with one embodiment;

FIG. 4B illustrates a photomicrograph of a fat replica comprising alginate, croconate oil, and sorbitan 80 in accordance with one embodiment; and

FIG. 4C illustrates a photomicrograph of a fat replica comprising alginate, sunflower oil, and sorbitan 80 in accordance with one embodiment.

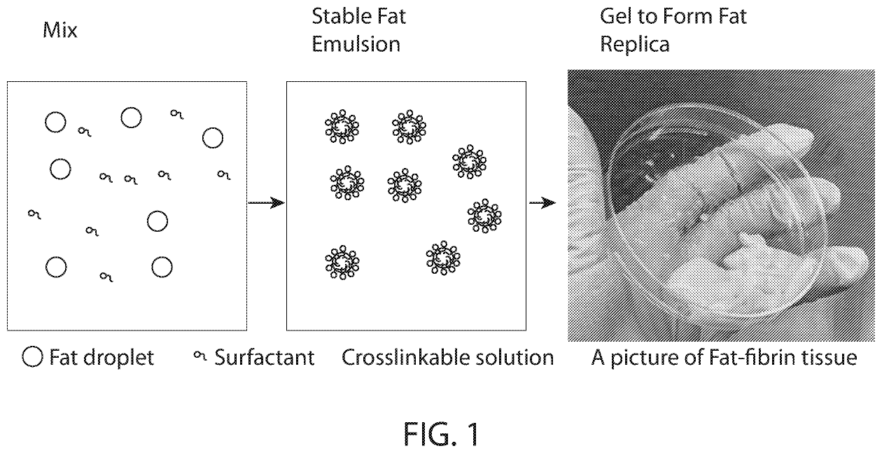

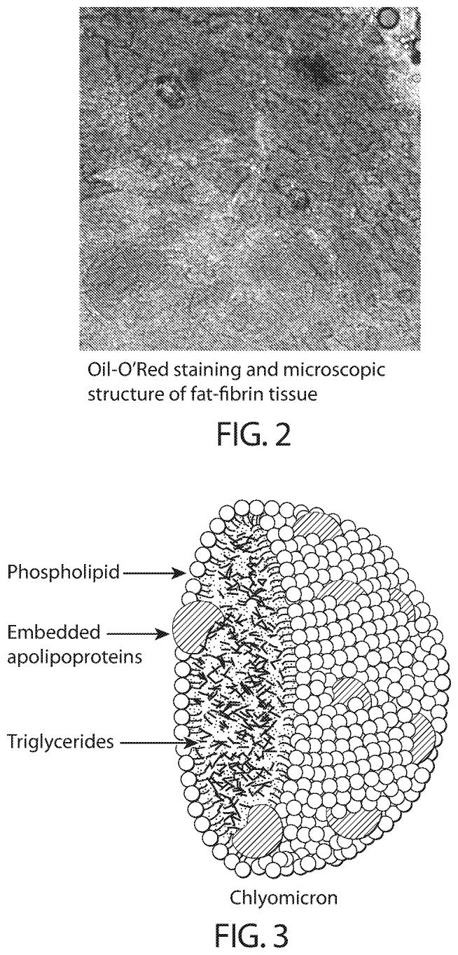

### DETAILED DESCRIPTION

The present disclosure generally relates, in certain aspects, to cultivated meat and other cultivated animal-derived products. In some embodiments, such products may include fat replicas, which may improve taste, appearance, etc. In some cases, a fat replica can be formed by forming a fat emulsion in non-human blood plasma, then causing the blood plasma to crosslink and/or clot, e.g., forming a hydrogel containing the fat emulsion. In some cases, the fat replica may be used to make a cultivated meat product, e.g., by combining with muscle replicas, lysate of non-human red blood cells, etc. Other embodiments are generally directed to methods of making or using such fat replicas, the microcarriers, or the cultivated meat products, kits involving these, or the like.

Cultivated meat is often described using terms such as cultured meat, tissue mass, cellular (or cell-based) meat, slaughter-free meat, and synthetic meat, among other related terms. One aspect of the present disclosure is generally directed to a cultivated meat product that includes a fat replica, e.g., fat that is artificially added to the product, as opposed to “regular” fat that naturally occurs in the meat grown and harvested from live animals.

For example, in one embodiment, the fat replica may comprise fat and a hydrogel. The fat may arise from any suitable source, or more than one source in some instances. For example, the fat may arise from animal cells and/or plants. Examples of plant-based fats include vegetable oil, corn oil, and other oils such as those described herein. Animal-based fats may, in some embodiments, be formed from fat (or adipose) cells that are cultivated, for example, on microcarriers, or other suitable scaffolds. The microcarriers or other scaffolds may comprise fibrin and/or other suitable materials such as those discussed below. In some cases, the fat cells may be grown in a reactor, such as those described herein. As yet another example, the fat may include fat that was synthetically prepared.

In one set of embodiments, the fat in the fat replica may be present in an emulsion. An emulsion of fat may be prepared, for example, by emulsifying fat with non-human blood plasma. In some cases, the fat may be caused to form a fat emulsion by mixing the fat with non-human blood plasma. Without wishing to be bound by any theory, it is believed that the plasma has components that can emulsify fat to form fat particles such as chylomicrons, e.g., as shown in FIG. 3. For instance, the plasma may include proteins or surfactants that can from such fat particles.

The non-human blood plasma may be treated in some embodiments to form a fat replica. For example, fibrin within the plasma may be caused to clot and/or by causing the fibrin to crosslink, e.g., by exposing it to thrombin, calcium, or other clotting agents such as those described herein. In addition, in some embodiments, a fat replica may comprise a fat emulsion contained within a hydrogel. The hydrogel may be formed from non-human blood plasma, e.g., as discussed, and/or another component. Non-limiting examples of such hydrogels include alginate, gelatin, or others such as those described herein.

Fat is typically an integral part of naturally-occurring meat, and exists in muscle tissue in varying percentages. Its presence can be useful in causing a cultivated meat product to taste more like naturally-occurring meat. Thus, as discussed herein, various fat replicas are provided in certain embodiments, which may be added to a cultivated meat product, or other cultivated animal-derived products such as those disclosed herein.

Certain structures and methods described herein can be useful, for example, by providing meat and other animal derived products for human consumption. Certain embodiments of structures and methods described herein may offer certain advantages as compared to existing agriculture-based methods of meat production, for example, by significantly reducing the number of animals bred for slaughter, thus decreasing the number of foodborne illnesses, diet related diseases, and the incidence of antibiotic resistance and infectious disease (e.g., zoonotic diseases such as Nipah virus and influenza A). In some cases, reducing the number of livestock worldwide may also have an effect on the environmental risks associated with agricultural farming due to, for example, ammonia emissions which contribute significantly to acid rain and acidification of ecosystems. In addition, in some instances, livestock, such as pigs and cows are a major agricultural source of greenhouse gases worldwide. In some embodiments, the structures and methods described herein may allow meat and other animal-derived products to be produced or cultivated in vitro, e.g., using blood and tissue donations obtained from living livestock donors (e.g., not intended for slaughter for human consumption). As a non-limiting example, certain embodiments as described herein are generally directed to a product comprising a muscle replica, a fat replica, and a lysate of red blood cell.

The above discussion describes non-limiting examples of certain aspects generally directed to fat replicas, e.g., containing fat and a hydrogel. These may be useful, for example, in cultivated meat products. Accordingly, more generally, various aspects are directed to various systems and methods for producing cultivated meat and other cultivated animal-derived products, e.g., containing a fat replica, such as discussed herein.

For example, certain aspects are generally directed to cultivated animal-derived products, such as cultivated meat, or other products. These may be produced, for example, using cells taken from an animal, but then the cells are cultured in vitro, e.g., using bioreactors, flasks, petri dishes, microwell plates, or other cell culture systems. Many cell culture systems will be known to those of ordinary skill in the art. This is in stark contrast to traditional techniques of sacrificing animals and harvesting their meat or other organs (e.g., skin, internal organs, etc.) for food or other uses. Although the original cells seeded to form the product may have originated or otherwise have originally been derived from a living animal, the bulk of the cells forming the actual product were grown or cultured in an in vitro setting, rather than naturally as part a living animal.

A variety of products may be formed from cells cultured in vitro. For instance, in certain embodiments, the products may form “cultivated meat,” or meat that is intended to be eaten, for example, by humans. It will be appreciated that, because it is to be eaten, such products will often be formed of edible or digestible materials, e.g., materials that can be digested, or degraded to form generally nontoxic materials within the digestive system. For instance, the cultivated meat may contain animal-derived cells (e.g., derived from a chicken, a cow, a pig, a sheep, a goat, a deer, a fish, a duck, a turkey, a shrimp, or other animals that are commonly recognized for widespread human consumption), such as fat cells, muscle cells, or the like. The cells may be wild-type or naturally-occurring cells (e.g., harvested from an animal), although in some embodiments, the cells may include genetically engineered cells, e.g., engineered in a way to increase proliferation. In addition, in some embodiments, the cultivated meat product may contain other edible materials, such as plant-originated materials. Non-limiting examples of edible materials include proteins, carbohydrates, sugars, saccharides, plant-based fats, etc., as well as polymers formed from these (for example, polylactic acid, polyglycolic acid, cellulose, etc.). In some cases, the edible materials may be digested to form nutrients, e.g., such as amino acids, sugars, etc. that have nutritional value, for example, when taken up into the body. However, in some cases, the edible materials cannot be digested, and/or can be digested to form non-nutrients that cannot be absorbed as nutrients, but can be passed through the digestive system without detrimental effects.

In addition, it should be understood that the present disclosure is not limited to only cultivated meat products. In some cases, products such as those described herein may be cultivated from animal-derived cells, but the product is not necessarily one that is intended to be eaten. For instance, cells from an animal may be cultured to form various organs that can be harvested, such as skin, hair, fur, or the like. Thus, as a non-limiting example, leather, cultivated fur, etc. can be formed by growing cells in culture, for example as discussed herein, without the traditional method of sacrificing animals to harvest their skin or other organs.

In one set of embodiments, the cultivated meat product may contain a fat replica. As discussed, a fat replica is not “regular” fat that is naturally grown within a live animal. Instead, the fat replica may be artificially produced through a variety of techniques. For example, in one embodiment, the fat replica comprises one or more plant-based fats. Plant-based fats can include any fat obtained or extracted from a plant, e.g., vegetable oil, sunflower seed oil, corn oil, safflower oil, oleic acid, canola oil, omega-3 fatty acids, omega-6 fatty acids, olive oil, peanut oil, palm oil, cocoa butter, coconut oil, rapeseed oil, linseed oil, almond oil, sesame oil, soybean oil, etc. As another example, the fat replica may comprise one or more animal fats, although at least some of the animal fats may be produced artificially, e.g., using in vitro techniques such as those described herein. For example, as discussed below, the fat may contain fat cells grown within a bioreactor or other cell culture systems. Even though the fat cells may have originated from an animal, the bulk of the fat cells forming the fat replica may be grown or cultured in an in vitro setting, rather than naturally as part a living animal.

The fat replica may be produced to have any of a variety of characteristics. For example, the fat replica may be present within a cultivated meat product, or other cultivated animal-derived product, and it may be desirable for the fat replica to replicate certain characteristics of naturally occurring fat. These may include, for example, content, textures, tastes, mechanical properties, rheological properties (e.g., elastic modulus, loss modulus, etc.), etc. In addition, in some embodiments, the fat replica may include certain types of fat that are desirable or beneficial. For instance, the fat replica may be enriched in certain types of fat that are perceived to be desirable for a particular application. For instance, the fat replica may be enriched or predominantly contain plant-derived fats, saturated or unsaturated fats, or the like.

Thus, in some embodiments, the fat may include an animal fat, a plant fat, or both. In certain embodiments, the fat may include saturated fat, unsaturated fat, or both. In one set of embodiments, a fat replica may comprise non-human fat cells, such as adipose cells, adipose progenitor cells, etc., and/or other types of fat cells. The cells may come from any suitable animal, such as a chicken, a cow, a pig, a sheep, a goat, a deer, a fish, a duck, a turkey, a shrimp, or the like. In some embodiments, the cells may be seeded onto microcarriers, or other scaffolding material. A variety of microcarriers can be used, for example microcarriers comprising fibrin, such as those discussed below. Microcarriers are also discussed in U.S. Ser. No. 63/159,403, filed Mar. 10, 2021, entitled “Constructs for Meat Cultivation and Other Applications,” by Khademhosseini, et al.; and a patent application entitled “Constructs Comprising Fibrin or Other Blood Products for Meat Cultivation and Other Applications,” filed on Nov. 15, 2021, U.S. Pat. Apl. Ser. No. 63/279,617, each incorporated herein by reference in its entirety. In certain cases, the cells may be cultured in a bioreactor or other in vitro cell culture system, such as described herein, to form a fat replica. In certain embodiments, for example, the cells are grown in serum, and induced to differentiate.

In some embodiments, the fat may be present within the fat replica as an emulsion. For example, the fat may be contained within fat particles in the fat replica. In some cases, the fat may be dispersed (for example, homogenously) within the emulsion. In one set of embodiments, for example, fat particles may be formed by mixing fat with non-human blood plasma, which are suspended as an emulsion. The non-human blood plasma may come from any suitable source. As non-limiting examples, the plasma may arise from the blood of an animal, such as a chicken, a cow, a pig, a sheep, a goat, a deer, a fish, a duck, a turkey, a shrimp, etc. The plasma may arise from the same, or a different type of animal than the fat or fat cells that may be present. As previously discussed, and without wishing to be bound by any theory, it is believed that the plasma has components that can emulsify fat to form fat particles. Fat particles that may be formed include chylomicrons, very low density lipoproteins, low density lipoproteins, high density lipoproteins, and the like. These may be formed from various proteins, surfactants, etc. present with in the plasma. For example, particles such as chylomicrons may comprise apolipoproteins such as A1, A2, A3, A4, A5, B, B48, B100, C1, C2, C3, C4, D, E, F, H, L, M, or the like. Apolipoproteins such as these may be present within the plasma, e.g., naturally-occurring within the plasma. In some embodiments, however, various proteins, surfactants, or the like may be added. In some embodiments, the volumetric ratio of fat to non-human blood plasma may be at least 10:90, at least 20:80, at least 30:70, at least 40:60, at least 50:50, at least 60:40, at least 70:30, at least 80:20, at least 90:10, etc.

In addition, in certain embodiments, a surfactant may be used to disperse the fat and/or stabilize the emulsion. Non-limiting examples of surfactants include phospholipids, monoglyercols, diglycerols, propylene glycol monoesters, lactylate esters, polyglycerol esters, sorbitan esters, ethoxylated esters, succinate esters, fruit acid esters, acetylated monoglycerols, acetylated diglycerols, phosphate monoglycerols, phosphate diglycerols, sucrose esters, etc. For example, a surfactant may be mixed with animal cells and/or non-human blood plasma to form an emulsion.

Non-human blood plasma may be edible. In some cases, the non-human blood plasma may contain fibrinogen, which can be used to form fibrin hydrogels as discussed herein, for example, by the addition of thrombin or calcium. In some embodiments, the non-human blood plasma may be concentrated or diluted, for example, to increase or decrease the crosslinking density of the hydrogel. Accordingly, in another set of embodiments, the fat replica may comprise a hydrogel. Fat may be present within the hydrogel, e.g., suspended within a fluid contained within the hydrogel, e.g., as an emulsion and/or present as fat particles, dissolved or suspended within the fluid, etc. Such a system may form a fat replica that can be used within a cultivated meat product, or other cultivated animal-derived product such as those described herein.

In one set of embodiments, the hydrogel may be formed, at least in part, by fibrin. Fibrin is a fibrous protein involved in the clotting of blood. It can be formed, for example, by exposing a non-human blood plasma to a clotting agent. For instance, the protease inhibitor thrombin is able to act on fibrinogen, causing the fibrinogen to form fibrin and thereby form a clot. The clot may be edible. Other techniques can also be used to crosslink fibrin in certain cases, e.g., artificially, rather than causing the clotting process to occur. For example, in one set of embodiments, calcium may be added to cause fibrin to crosslink. Thus, in certain embodiments, a fat replica may comprise a hydrogel comprising fibrin, e.g., that has been clotted to form the hydrogel. In other embodiments, the fibrin may not be clotted, but may form a hydrogel, e.g., by inducing crosslinking of the fibrin, for example, chemically.

However, it should be understood that the fat replica is not limited to only fibrin hydrogels. The hydrogel may be formed from other components, in addition to or instead of fibrin. Non-limiting examples of hydrogels include proteins (for example, collagen, gelatin, etc.), polymers (for example, polylactic acid, polyglycolic acid, etc.), and carbohydrates (for example, alginate, hyaluronan, chitosan, cellulose, hydroxymethyl cellulose etc.). In some cases, a hydrogel may be formed by causing components such as these to crosslink, e.g., in the presence of fat, to form a fat replica.

The hydrogels can be non-covalently and/or covalently crosslinked. Non-covalent hydrogels may be stabilized in some embodiments by hydrogen bonding, van der Waals interactions (e.g., hydrophobic interactions), etc. Covalent hydrogels may be formed, for example, by adding a crosslinking agent, bearing a first coupling group, to a crosslinkable material, bearing a second coupling group. The coupling groups can be any functional groups known to those of skill in the art that together form a covalent bond, for example, under mild reaction conditions or physiological conditions. Examples of couple groups include, but are not limited to, maleimides, N-hydroxysuccinimide (NHS) esters, carbodiimides, hydrazide, pentafluorophenyl (PFP) esters, phosphines, hydroxymethyl phosphines, psoralen, imidoesters, pyridyl disulfide, isocyanates, vinyl sulfones, alpha-haloacetyls, aryl azides, acyl azides, alkyl azides, diazirines, benzophenone, epoxides, carbonates, anhydrides, sulfonyl chlorides, cyclooctynes, aldehydes, and sulfhydryl groups, etc. In some embodiments, coupling groups may include free amines (—NH₂), free sulfhydryl groups (—SH), free hydroxide groups (—OH), carboxylates, hydrazides, alkoxyamines, etc. In some embodiments, a coupling group can be a functional group that is reactive toward sulfhydryl groups, such as maleimide, pyridyl disulfide, or a haloacetyl.

In addition, it will be understood that the characteristics of the fat replica can be controlled, to some extent, by controlling the crosslinking density of the hydrogel, or other techniques such as those described herein. For example, characteristics such as texture, mechanical properties, rheological properties (e.g., elastic modulus, loss modulus, etc.), or the like may be controlled by controlling the crosslinking or hydrogel formation conditions and/or the components of the hydrogel. In addition, in some cases, the characteristics of the hydrogel may be controlled based on the type of non-human blood plasma used.

For example, in some embodiments, the crosslinking density may vary, e.g., by altering the concentration of the fibrinogen in the non-human blood plasma. In some embodiments, the crosslinking density may be determined, for example, using measurements such as rheological measurements, dynamical mechanical analysis measurements, or the like. In some embodiments, higher crosslinking densities may be useful for certain applications, e.g., for mimicking higher fat contents (e.g., more tender meat products). In certain embodiments, lower crosslinking densities may be useful for certain applications, e.g., mimicking lower fat contents (e.g., leaner meat products) or looser fat. Rheological measurements, e.g., the storage modulus, the loss modulus, etc., may be used to determine the characteristics of the fat replica and/or cultivated meat product (or other product). Examples of such characteristics include, but are not limited to textual proprieties, microstructural proprieties, rheological proprieties, or the like. These may be determined, for example, using rheometers or other techniques known to those of ordinary skill in the art.

In certain embodiments, the fat replica may be formed by seeding fat cells such as adipose cells, or adipose progenitor cells, onto microcarriers, and culturing them in a bioreactor to form the fat replica. In some cases, fibrin may be used as a scaffolding material, for example, formed as microcarriers. Fibrin is a fibrous protein involved in the clotting of blood. It can be formed by the action of the protease inhibitor thrombin on fibrinogen, which causes it to polymerize and form a clot. Fibrin can be used as a passive scaffolding material in some embodiments. However, in some embodiments, fibrin can specifically bind certain growth factors in the cell culture media that promote cell adhesion, proliferation, and migration. Non-limiting examples include fibronectin, hyaluronic acid, von Willebrand factor, or the like.

For example, cells derived from an animal may be seeded onto microcarriers or scaffolds, and grown in vitro, e.g., in a bioreactor or other cell culture systems such as are described herein, to produce a cultivated product. In some cases, the product thus formed can be used without additional processing. As a non-limiting example, a cultivated meat product may be grown by seeding fat cells on microcarriers or scaffolds, then growing them within a bioreactor.

In certain embodiments, microcarriers or scaffolds such as those discussed herein may be treated to facilitate binding of cells, such as fat cells. For example, the microcarriers or scaffolds may be exposed to non-human serum, which may include growth factors that bind to the microcarriers or scaffolds. The growth factors may, for example, promote cell adhesion, proliferation, and/or migration of cells into the microcarriers or scaffolds. In addition, in some cases, the microcarriers or scaffolds may have structures, such as grooves, that may allow the cells such as myoblasts to become aligned in a specific direction, although this is not a requirement. Such structures are described in U.S. Ser. No. 63/159,403, filed Mar. 10, 2021, entitled “Constructs for Meat Cultivation and Other Applications,” by Khademhosseini, et al., incorporated herein by reference in its entirety.

In some embodiments, the microcarriers or scaffolds may comprise any material that forms an edible hydrogel, such as fibrin. For example, in one embodiment, a microcarrier may be formed from a non-human blood plasma which contains plasma-rich fibrinogen that can be crosslinked or otherwise processed to form a fibrin hydrogel. Such crosslinking can be achieved by exposure to thrombin, calcium, or other conditions such as those described herein. In some embodiments, fibrin hydrogels are formed using non-human blood plasma containing fibrinogen, e.g., at least 10 wt %, or more in some cases.

In certain embodiments, non-human cells such as adipose cells may be seeded on the microcarriers or other scaffolds, and grown in a bioreactor or other in vitro cell culture system.

In one set of embodiments, a scaffold may have a largest or maximum internal dimension of less than 100 mm, less than 80 mm, less than 70 mm, less than 60 mm, less than 50 mm, less than 40 mm, less than 30 mm, less than 20 mm, less than 10 mm, less than 5 mm, less than 3 mm, less than 2 mm, or less than 1 mm. In addition, in some cases, the microcarriers may have a maximum internal dimension that is at least 1 mm, at least 2 mm, at least 3 mm, at least 5 mm, at least 10 mm, at least 20 mm, at least 30 mm, at least 40 mm, at least 50 mm, at least 60 mm, at least 70 mm, at least 80 mm, at least 90 mm, at least 100 mm, etc. Combinations of any of these dimensions are also possible in some embodiments.

The scaffold may comprise any suitable material. For example, in one set of embodiments, the scaffold may comprise fibrin, or another edible material. This may be useful for applications such as cultivated meat, where the cultivated animal-derived product will be eaten, e.g., by humans or other animals. In some embodiments, the microcarriers may comprise a hydrogel, e.g., a fibrin hydrogel, or other hydrogels such as those described herein.

In some cases, at least 50 wt %, at least 60 wt %, at least 70 wt %, at least 80 wt %, at least 90 wt %, or substantially all of a scaffold is formed from fibrin, and/or another edible material. The fibrin may arise from any suitable source. For example, the fibrin may arise from a non-human animal, such as a non-human mammal. Non-limiting examples include cows, pigs, sheep, goats, or the like. In some cases, the fibrin may arise from the blood of such an animal. For instance, in some embodiments, the fibrin may be prepared by acquiring blood or blood plasma from an animal, and processing it to produce fibrin. For example, in one set of embodiments, the blood is exposed to a protease inhibitor such as thrombin, which may cause fibrinogen to clot to form fibrin. The fibrin may be harvested, and used as discussed herein, e.g., to produce scaffolds such as microcarriers. In addition, in some cases, fibrin may be obtained from fibrinogen, which may be bought commercially, obtained from blood plasma, or the like.

In some cases, the blood may be acquired from the animal without killing the animal. For instance, blood may be withdrawn from the animal at spaced intervals, so as to allow the animal time to recover and produce new blood. For instance, blood may be withdrawn from the animal every 4 weeks, every 6 weeks, every 2 months, or the like. Additional details may be found in a patent application entitled “Methods and Systems of Preparing Cultivated Meat from Blood or Cellular Biomass,” filed on Nov. 15, 2021, U.S. Pat. Apl. Ser. No. 63/279,631, incorporated herein by reference in its entirety.

The fibrin may be processed to form a scaffold. In one set of embodiments, the scaffold may take the form of one or more microcarriers. The microcarriers may have any shape or size. In some cases, more than one type of microcarrier may be present, e.g., some of which may have various materials, shapes, sizes, etc., such as are described herein. For example, in some embodiments, at least some of the microcarriers may be substantially spherical or exhibit spherical symmetry, although in other embodiments, at least some of the microcarriers may be non-spherically symmetric (for example, triangular) or may be anisotropic. In addition, in certain cases, at least some of the microcarriers may have a plurality of grooves, e.g., as discussed herein.

In certain embodiments, the microcarriers may have a largest or maximum internal dimension of less than 100 mm, less than 80 mm, less than 70 mm, less than 60 mm, less than 50 mm, less than 40 mm, less than 30 mm, less than 20 mm, less than 10 mm, less than 5 mm, less than 3 mm, less than 2 mm, less than 1 mm, less than 0.5 mm, less than 0.3 mm, less than 0.2 mm, less than 0.1 mm, less than 0.05 mm, less than 0.03 mm, less than 0.02 mm, or less than 0.01 mm. In addition, in some cases, the microcarriers may have a maximum internal dimension that is at least 0.01 mm, at least 0.02 mm, at least 0.03 mm, at least 0.05 mm, at least 0.1 mm, at least 0.2 mm, at least 0.3 mm, at least 0.5 mm, at least 1 mm, at least 2 mm, at least 3 mm, at least 5 mm, at least 10 mm, at least 20 mm, at least 30 mm, at least 40 mm, at least 50 mm, at least 60 mm, at least 70 mm, at least 80 mm, at least 90 mm, at least 100 mm, etc. In addition, in certain cases, combinations of any of these dimensions are also possible. As non-limiting examples, the microcarriers may have a maximum internal dimension of between 10 mm and 30 mm, between 5 mm and 20 mm, between 3 mm and 10 mm, between 50 mm and 70 mm, between 1 mm and 3 mm, etc. In some cases, the maximum internal dimension is the length of longest straight line that can be contained entirely within the microcarrier and/or the interior of the microcarrier (e.g., if the microcarrier defines a hollow sphere).

In some cases, however, some or all of the microcarriers may not necessarily be spherical. For example, at least some of the microcarriers may have shapes such as cubical, rectangular solid, triangular, tetrahedral, octahedral, irregular, etc. In some cases, at least some of the microcarriers have a shape that is substantially planar. For instance, the microcarrier may have a generally rectangular shape where the smallest dimension of the rectangular solid is substantially smaller than either of the other two dimensions, for example, by a factor of at least 3, at least 5, or at least 10, etc.

In addition, in certain cases, at least some of the microcarriers have a relatively large surface to volume ratio. This may be important, for example, in embodiments where the microcarriers contain a plurality of grooves, e.g., as discussed herein. In contrast, a perfect sphere would have the smallest possible surface to volume ratio for a given volume of material. As a nonlimiting example, the surface to volume ratio may be at least 100, at least 200, at least 300, etc., e.g., for a sheet thickness of 0.01 mm surface and an area of 1 mm×10 mm.

Fibrin itself may be edible. The microcarrier or scaffold may comprise, in addition to or instead of fibrin, other edible materials in certain embodiments. In addition, it should be understood that the scaffold is not limited to only edible or degradable materials. In other embodiments, the scaffold may comprise materials, such as polymers, that are not necessarily edible and/or degradable. Non-limiting examples of such materials include natural polymers such as proteins (e.g., silk, collagen, gelatin, fibrinogen, elastin, keratin, actin, myosin, etc.), polysaccharides (e.g., cellulose, amylose, dextran, chitin, glycosaminoglycans), or the like. Other examples include polymers such as polylactic acid, polyglycolic acid, poly(lactic-co-glycolic acid), polyhydroxyalkanoates, polycaprolactones, etc., bioactive ceramics such as hydroxyapatite, tricalcium phosphate, silicates, phosphate glasses, glass-ceramic composites (such as apatite-wollastonite), etc., or the like.

While almost anything can physically be eaten, materials that are edible include those that are found naturally occurring in foods that are commonly eaten by significant percentages of the general population. Examples of edible materials include, but are not limited to proteins or peptides, polysaccharides, carbohydrates, or the like. In some cases, such materials may be broken down by the digestive system to produce nutrients such as amino acids, monosaccharides, simple sugars, etc. However, in some cases, the edible materials need not be digestible into such nutrients. Specific non-limiting examples of edible materials include cellulose, chitin, collagen, soy protein, mycelium, gelatin, alginate, etc. Additionally, in some but not all embodiments, the scaffold may comprise a plant-originated material, such as a plant-originated protein. Such plant-originated materials may be harvested directly from a plant, be grown in vitro (e.g., in cell culture from a culture initially originating in a plant), be synthetically produced (e.g., without using a plant, e.g., chemically produced), etc. Examples of protein-originated material include, but are not limited to, cellulose or certain proteins, such as prolamin, zein, fibrin, gliadin, hordein, secalin, kafirin, avenin, gliadine, 2S albumin, globulin, glutelin, etc. The plant that material originates from may be any plant, including but not limited to food crop plants. Non-limiting examples of plants include, but are not limited to, wheat, barley, rye, corn, sorghum, oats, quinoa, hemp, potato, soy, etc. Additional examples of such materials include those described in U.S. Ser. No. 63/159,403, filed Mar. 10, 2021, entitled “Constructs for Meat Cultivation and Other Applications,” by Khademhosseini, et al., incorporated herein by reference in its entirety.

The microcarrier, or other scaffold may also be biocompatible in some instances. In addition, in certain embodiments, the scaffold may comprise a polymer, e.g., one that is biodegradable. For example, in some cases, the scaffold may be one that begins to spontaneously degrade (for example, via hydrolysis reactions, dissolution, etc.) when maintained in contact with water, e.g., for at least 12 hours.

The microcarriers, or other scaffolds such as described herein, may be formed using any suitable technique, according to certain aspects. Non-limiting examples include extrusion, electrospinning, 3D-printing, molding, injection molding, or the like, e.g., of a precursor solution or a hydrogel block, etc. For example, a microcarrier or other scaffold may be formed by milling, chopping, or otherwise processing hydrogel blocks. As still another non-limiting example, fibrin hydrogel blocks can be formed into millimeter-sized microcarriers using high speed homogenizers, or the like. In yet another other non-limiting example, cells may be confined on an engineered surface or material having a micro-nanotopography as contact guidance, or by applying mechanical forces generated either by the contractile activity of the cells or by an external strain.

In one set of embodiments, materials that will be used to form a microcarrier (e.g., comprising fibrin, etc.) may be formed into a paste or other mixture that is extruded, e.g., at low temperatures (e.g., temperatures below 20° C., 15° C., 10° C., or 5° C., etc.) and/or into a water bath to solidify and/or coagulate the materials into microcarriers. The mixture may have, for example, at least 20%, at least 30%, at least 40%, at least 50%, at least 60%, or at least 70% material (e.g., fibrin), and/or no more than 80%, no more than 70%, no more than 60%, no more than 50%, no more than 40%, or no more than 30% material, by weight. In some cases, combinations of any of these ranges are also possible, e.g., the mixture may have between 40% and 60% material (e.g., fibrin), between 20% and 80% material, between 30% and 50% material, etc. In some cases, the materials may be dissolved and/or suspended in a suitable liquid, e.g., water, a strong alcohol (e.g., 70% to 80% aqueous solution by volume), an acid solution, an alkaline solution, or the like. These percents are percent by weight.

In some embodiments, the microcarriers (or other scaffolds) may be formed to have any of a wide variety of shapes, such as flakes, plates, fibers, whiskers, or the like, e.g., having dimensions such as any of those described herein. In addition, as previously noted, in certain embodiments, some of these shapes may contain grooves.

As a non-limiting example, the microcarriers may have the form of fibers, e.g., having an average length of at least 1 micrometer, at least 2 micrometers, at least 3 micrometers, at least 4 micrometers, at least 5 micrometers, at least 10 micrometers, at least 20 micrometers, at least 30 micrometers, at least 40 micrometers, at least 50 micrometers, at least 100 micrometers, at least 200 micrometers, at least 300 micrometers, at least 400 micrometers, at least 500 micrometers, at least 1 mm, at least 2 mm, at least 3 mm, at least 4 mm, at least 5 mm, at least 1 cm, at least 2 cm, at least 5 cm, at least 10 cm, at least 20 cm, at least 30 cm, at least 50 cm, etc. In addition, in certain embodiments, the fibers may have an average length of no more than 100 cm, no more than 50 cm, no more than 30 cm, no more than 20 cm, no more than 10 cm, no more than 5 cm, no more than 4 cm, no more than 3 cm, no more than 2 cm, no more than 1 cm, no more than 5 mm, no more than 4 mm, no more than 3 mm, no more than 2 mm, no more than 1 mm, no more than 500 micrometers, no more than 400 micrometers, no more than 300 micrometers, no more than 200 micrometers, no more than 100 micrometers, etc. Combinations of any of these are also possible, e.g., the fibers may have an average length of between 200 micrometers and 500 micrometers, between 500 micrometers and 5 mm, between 300 micrometers and 1 mm, between 10 micrometers and 400 micrometers, etc.

In some cases, the scaffold or microcarrier containing cells may be usable with the scaffold or microcarrier in place. For example, the cells and the scaffold or microcarrier together may form a cultivated meat product (for example, if the scaffold or microcarrier is edible and/or degradable), or other cultivated animal-derived product. In some embodiments, the non-human cell-to-microcarrier ratio in the product is at least 95:5, at least 85:15, at least 75:25, at least 25:75, at least 15:85, or at least 5:95. However, in some embodiments, the cells may be separated from the scaffold or microcarrier. For example, the scaffold or microcarrier may be removed and reused or discarded, while the cells may be used without the scaffold present. Thus, for example, if the cells are used in a cultivated meat product or other cultivated animal-derived product, the scaffold or microcarrier may not necessarily be edible and/or degradable.

In some embodiments, a cultivated meat product may be formed by mixing a fat replica (e.g., comprising a fat emulsion and non-human blood plasma), and a lysate of non-human red blood cells. In some embodiments, the non-human cell to fibrin microcarrier ratio is at least 95:5, at least 85:15, at least 75:25, at least 25:75, at least 15:85, at least 5:95, etc. In certain other embodiments, the percent by weight of fat replica to muscle replica is at least 5:95, at least 10:90, at least 15:85, at least 20:80, at least 30:70, etc.

In some instances, a product such as a cultivated meat product further comprises binding agents that hold the various components together. Exemplary embodiments include transglutaminase, non-human plasma, fibrinogen, soy isolate, a soy concentrate, a soy milk, an egg, a soy flour, a wheat gluten isolate, or a pea isolate.

In some cases, the microcarriers (or other scaffolds) may be purified, e.g., by extracting impurities prior to use, e.g., prior to seeding with cells. For example, impurities such as citric acid or ethanol may interfere with cell culture, and/or may interfere with the taste of the cultivated meat product. Thus, in some cases, such microcarriers or other scaffolds may be exposed to water, e.g., washed, to remove potential contaminants.

In addition, in certain embodiments, the microcarriers or other scaffolds may be sterilized before use, e.g., prior to seeding with cells. A variety of techniques for sterilizing the microcarriers can be used, including but not limited to, applying ultraviolet light or high temperatures (e.g., a temperature of at least 100° C.) to the microcarriers. Those of ordinary skill in the art will be aware of various sterilization techniques that may be used.

Certain other aspects are directed toward methods of forming fat replicas. In certain embodiments, for example, a fat replica can be formed by dispersing fat, optionally in the presence of a surfactant, in a solution, and inducing crosslinking, for example to cause the formation of a clot and/or a hydrogel containing the fat. For example, the fat source may be acellular or comprise cells. The fat may arise from any suitable source, for example, an animal, a plant, etc.

In some embodiments, the fat replica may be incorporated within a cultivated meat product, or other product such as those described herein. In some embodiments, the fat replica may be added to other materials to make a cultivated meat product, or other cultivated animal-derived products. For example, a fat replica may be combined with non-human animal cells, such as myoblasts, to form a cultivated meat product.

As a non-limiting example, in some cases, the fat replica is mixed with non-human myoblasts, which may be seeded on microcarriers in some embodiments. Examples of microcarriers include microcarriers comprising fibrin, or other microcarriers, including those discussed in a patent application entitled “Constructs Comprising Fibrin or Other Blood Products for Meat Cultivation and Other Applications” filed on Nov. 15, 2021, U.S. Pat. Apl. Ser. No. 63/279,617, incorporated herein by reference in its entirety. The non-human animal cells may, for example, form a muscle replica. In addition, the non-human animal cells may arise from the same or different animals as any fat or fat cells present within the fat replica. In some other embodiments, the tissue mass has a ratio of cultured cells to the fat emulsion of at least 5:95, of at least 10:90, of at least 15:85, of at least 20:80, of at least 30:70, etc.

Some embodiments, for instance, are directed to the assembly of the final cultivated meat product. For instance, in some embodiments, the cultivated meat product is formed by mixing the fat replica with the muscle replica. In another embodiment, the cultivated meat product is formed by lysing non-human red blood cells to produce a cell lysate and mixing with non-human muscle cells on microcarriers comprising fibrin.

In some cases, relatively large quantities of product may be prepared, e.g., by growing the cells in a bioreactor or other in vitro cell culture system, until at least a certain size or mass is reached. For example, the cells may be grown until they form a product that is, for example, at least 10 g, at least 25 g, at least 50 g, at least 100 g, at least 300 g, at least 1 kg, etc. Those of ordinary skill in the art will be aware of bioreactors and other cell culture systems.

In yet another embodiment, a product such as a cultivated meat product may be mixed with the lysate of non-human red blood cells to impart the cultivated meat product with the red appearance of native red muscle. In some cases, at least 1%, at least 3%, at least 5%, at least 10%, at least 20%, of the product comprises the lysate of non-human red blood cells. In some embodiments, the non-human red blood cells are lysed within 24 hours of withdrawal from a non-human living donor. See, e.g., a patent application entitled “Methods and Systems of Preparing Cultivated Meat from Blood or Cellular Biomass,” filed on Nov. 15, 2021, U.S. Pat. Apl. Ser. No. 63/279,631, incorporated herein by reference.

As mentioned, in some aspects, a product such as a cultivated meat product may be produced within a bioreactor or other cell culture system. A wide variety of bioreactors can be used in various embodiments including, but not limited to, suspension bioreactors, continuous stirred-tank bioreactors, rocker bioreactors, airlift bioreactors, fixed bed bioreactors, bubble column bioreactors, fluidized bed bioreactors, packed bed bioreactors, or the like.

In some cases, cells may be seeded on microcarriers or other scaffolds, then introduced into the bioreactor or other cell culture system. Those of ordinary skill in the art will be familiar with techniques for seeding cells on a scaffold. For instance, the scaffold may be exposed to a suspension containing animal-derived cells, which are allowed to settle from the suspension onto the scaffold. In some cases, one or more than one type of cell may be present in suspension and allowed to settle.

In some cases, a product can be formed within the bioreactor without additional processing, for example, without separating the cells or tissues grown within the bioreactor. However, in other cases, some separation and/or processing of the cells may be used. As a non-limiting example, myotubes may be grown within a bioreactor or other cell culture system such as those described herein to produce a muscle replica. In some embodiments, such muscle replicas may be processed, e.g., by adding a fat replica to produce a cultivated meat product having any desired ratio of muscle to fat in it. For instance, the ratio of muscle to fat may be at least 95:1, at least 90:1, at least 70:1, at least 50:1, at least 30:1, at least 20:1, at least 10:1, at least 5:1, at least 1:1, etc. by weight.

A variety of techniques may be used to grow cells within the bioreactor or other cell culture system. For instance, the cells may be grown at body temperature (e.g., about 38.5° C. for cow cells, about 41° C. for chicken cells, about 39-40° C. for pig cells, about 40-42° C. for duck cells, etc.). In some embodiments, during cultivation, the cells may have a shear stress applied to them of at least 0.005 newton/meter squared, of at least 0.01 newton/meter squared, of at least 0.2 newton/meter squared, of at least 0.3 newton/meter squared, of at least 0.4 newton/meter squared, of at least 0.5 newton/meter squared, of at least 0.6 newton/meter squared, of at least 0.7 newton/meter squared, of at least 0.8 newton/meter squared, etc.

In some embodiments, cells within the bioreactor or other cell culture system may be induced to differentiate, e.g., by adding suitable factors and/or altering the cell culture conditions therein. As a non-limiting example, myoblasts may be grown in serum, while removing or reducing the serum from the myoblasts may cause the myoblasts to differentiate to from myotubes. For instance, in one set of embodiments, the serum may be reduced from 10% to 2% to induce differentiation of myoblasts. Those of ordinary skill in the art will be aware of methods and systems to induce differentiation in cells.

In some embodiments, the serum can be obtained from commercial vendors. In certain cases, serum may be obtained from fresh whole blood. As a non-limiting example, the blood may be drawn within 24 hours from a living non-human animal donor, e.g., one that is not being slaughtered for meat. See, for example, “Methods and Systems of Preparing Cultivated Meat from Blood or Cellular Biomass,” filed on Nov. 15, 2021, U.S. Pat. Apl. Ser. No. 63/279,631, incorporated herein by reference in its entirety.

In one embodiment, the cell-based meat product may be grown in a bioreactor, or other in vitro cell culture system, comprising a cell growth medium. In one embodiment, the cell growth medium comprises an animal derived product, for example, platelet rich plasma (PRP), platelet poor plasma, platelet lysate (PL), platelet concentrate, a lysate of red blood cells, optionally comprising other nutrients, or the like. The cell growth medium may be used for the production of cell-based meat and/or to enhance the proliferation of primary cells, stem cells such as myoblasts, fibroblasts, adipocyte, vascular, osteoblasts, tenocyte, neural cells, etc. These cells may be isolated from human or non-human animals, grown in vitro, etc. These may include but are not limited to humans, cows, sheep, swine, horses, goats, camels, whales, fishes, crabs, shrimp and the like. In some embodiments, the blood products may be obtained from the blood of animals destined to slaughtered for food.

In one embodiment, the platelet rich plasma (PRP) may be derived from whole blood from which red blood cells and white blood cells have been removed, such as by centrifugation, filtration, or other techniques known to those of ordinary skill in the art. Platelet rich plasma (PRP) may be generally categorized based on its leukocyte and fibrin content as (1) leukocyte-rich PRP (L-PRP), (2) leukocyte reduced PRP (P-PRP); (3) leukocyte reduced/pure PRP, or (4) leukocyte platelet-rich fibrin/pure platelet-rich fibrin (L-PRF). The platelet-rich plasma may be a blood derived composition having an increased concentration of platelets, compared to normal blood. For example, the PRP may have at least double, at least five times, or at least ten times or more the normal concentration of platelets in blood. In addition, in accordance with another embodiment, the platelet rich plasma may contain a variety of endogenous growth factors, such as transforming growth factor beta, fibroblast growth factor, insulin-like growth factor 1, insulin-like growth factor 2, vascular endothelial growth factor, epidermal growth factor, Interleukin 8, keratinocyte growth factor, connective tissue growth factor, etc.

In one embodiment, the platelet concentrate (PC) may be derived from the platelet rich plasma (PRP), for example, by centrifugation. In some embodiments, the concentration may be at least 10³ platelets/mL, at least 10⁴ platelets/mL, at least 10⁵ platelets/mL, at least 10⁶ platelets/mL, at least 10⁷ platelets/mL, at least 10⁸ platelets/mL, at least 10⁹ platelets/mL, at least 10¹⁰ platelets/mL, etc. In some embodiments, donated platelet concentrates may be stored at 4° C. prior to use for at least 1 day, at least 2 days, at least 3 days, at least 4 days, at least 5 days, at least 6 days, or at least 7 days after donation. In another embodiment, expired human platelet concentrate may be obtained from blood banks, hospitals, and other institutions that routinely collect and store platelet rich plasma, and used as an additive in the cell growth medium.

In one embodiment, the platelet concentrate may impart the cell growth medium with antimicrobial properties. Platelets have certain properties similar to immune cells, and can in some cases induce potent anti-inflammatory responses when exposed to a number of chemical and biological triggers, for example, liposaccharide protein (LPS). In some embodiments, the platelet concentrate may be added to the cell culture medium and stimulated by treatment with platelet activating reagents, for example, calcium, thrombin, citrate, EDTA, plasminogen, and other platelet activating reagents known to those skilled in the art, e.g., to release antimicrobial molecules that may neutralize common bacterial, fungal, or viral food pathogens. In some embodiments, an acellular antimicrobial cell growth medium may be prepared by first culturing the platelet concentrate in the cell culture medium, stimulating them to release their antimicrobial payload, and then separating the antimicrobial cell growth medium from the platelet concentrate.

In one embodiment, the platelet concentrate may be lysed, for example by freeze-thawing or physical shearing (e.g. sonication or homogenization, etc.), to yield a platelet lysate (PL) comprising a plurality of cytokines and growth factors (e.g. transforming growth factor beta, fibroblast growth factor, insulin-like growth factor 1, insulin-like growth factor 2, vascular endothelial growth factor, epidermal growth factor, Interleukin 8, keratinocyte growth factor, connective tissue growth factor, etc.) that in some embodiments may enhance cell proliferation, for example, of myoblasts and adipocytes. In some embodiments, the platelet lysate comprises human platelets, and/or non-human platelets. For example, in one embodiment, the platelet rich plasma may include bovine platelet rich plasma.

In one embodiment, the cell growth medium comprises a combination of a platelet lysate (PL) and a platelet rich plasma (PRP). In one embodiment, the PL/PRP comprise at least 2 to 20% w/v, at least 5-15% w/v, or at least 10% w/v of the cell culture growth medium. In another embodiment, the total platelet component in the cell growth medium is at least 2 to 5 mg/mL, at least 2 to 10 mg/mL, at least 2 to 20 mg/mL, or at least 9 to 11 mg/mL.

The cells may be grown within the bioreactor or other cell culture system for any suitable length of time, e.g., to produce a cultivated product. For example, the cells may be grown for at least 3 days, at least 5 days, at least 1 week, at least 2 weeks, at least 3 weeks, at least 4 weeks, etc.

In some embodiments, a cultivated meat product may be formed by mixing a fat replica (e.g., comprising a fat emulsion and non-human blood plasma), non-human animal cells (e.g., as a muscle replica), and a lysate of non-human red blood cells. In some embodiments, the non-human cell to fibrin microcarrier ratio is at least 95:5, at least 85:15, at least 75:25, at least 25:75, at least 15:85, at least 5:95, etc. In certain other embodiments, the percent by weight of the muscle replica to fat replica is at least 95:5, at least 90:10, at least 80:20, at least 70:30, etc. In some embodiments, the fat to plasma ratio may be varied to alter the texture or toughness of the meat. For example, in one embodiment a fat to plasma ratio (volume basis) of 90:10 may render the fat tissue soft and pliable. In some embodiments, the fat to plasma ratio may be at least 95:5, at least 90:10, at least 80:20, at least 70:30, etc.

In some instances, a product such as a cultivated meat product further comprises binding agents that hold the various components together. Exemplary embodiments include transglutaminase, non-human plasma, fibrinogen, soy isolate, a soy concentrate, a soy milk, an egg, a soy flour, a wheat gluten isolate, or a pea isolate.

The following are each incorporated herein by reference in their entireties: U.S. Provisional Patent Application Ser. No. 63/159,403, filed Mar. 10, 2021, entitled “Constructs for Meat Cultivation and Other Applications”; U.S. Provisional Patent Application Ser. No. 63/279,617, filed Nov. 15, 2021, entitled “Constructs Comprising Fibrin or Other Blood Products for Meat Cultivation and Other Applications”; U.S. Provisional Patent Application Ser. No. 63/279,631, filed Nov. 15, 2021, entitled, “Methods and Systems of Preparing Cultivated Meat from Blood or Cellular Biomass”; U.S. Provisional Patent Application Ser. No. 63/279,642, filed Nov. 15, 2021, entitled, “Systems and Methods of Producing Fat Tissue for Cell-Based Meat Products”; U.S. Provisional Patent Application Ser. No. 63/279,644, filed Nov. 15, 2021, entitled “Production of Heme for Cell-Based Meat Products”; US Provisional Patent Application Serial No. U.S. 63/300,577, filed Jan. 18, 2022, entitled “Animal-Derived Antimicrobial Systems and Methods”; U.S. Provisional Patent Application Ser. No. 63/164,397, filed Mar. 22, 2021, entitled “Growth Factor for Laboratory Grown Meat”; U.S. Provisional Patent Application Ser. No. 63/164,387, filed Mar. 22, 2021, entitled, “Methods of Producing Animal Derived Products”; U.S. Provisional Patent Application Ser. No. 63/314,171, filed Feb. 25, 2022, entitled “Growth Factors for Laboratory Grown Meat and Other Applications”; and U.S. Provisional Patent Application Ser. No. 63/314,191, filed Feb. 25, 2022, entitled “Methods and Systems of Producing Products Such as Animal Derived Products.”

The following examples are intended to illustrate certain embodiments of the present disclosure, but do not exemplify the full scope of the disclosure.

### EXAMPLE 1

This example demonstrates the ability of fat emulsions stabilized within fibrin hydrogels to mimic the consistency and texture of fat content found in native meat products. Fat emulsions are made by adding a fat source to a solution of non-human blood plasma. In some embodiments, the fat source is an animal, a plant, or both. In certain embodiments, the fat source comprises a saturated fat, an unsaturated fat, or both. Non-human blood plasma can be derived from slaughtered animals or from living donors using apheresis by those skilled in the art and used to stabilize the fat. For example, blood plasma contains various globular proteins and macromolecules that can stabilize fat droplets following vigorous mixing to yield a homogenously dispersion which can be subsequently crosslinked to form a fat replica.

To demonstrate these principals, fresh bovine blood was obtained and immediately processed to separate the plasma from the cells. In one embodiment, sunflower oil was mixed with the bovine blood plasma at a volumetric ratio of 80:20 (fat:plasma), homogenized at 3000 rpm for 1 min minutes, and allowed to sit for 30 minutes at room temperature until gelation occurred. In another embodiment, thrombin or calcium may be added to the solution after homogenization to accelerate the gelation process.

In another embodiment, the fat to plasma ratio of the above system was varied from 90:10 to 10:90. It was observed that ratios with higher fat contents produced softer tissue constructs, for instance, when using and unsaturated fat. When saturated fats, such as coconut oil, were used; however, an increase in the fat ratio, particularly at lower temperature, increased the brittleness of fat tissue. In other embodiments, fat oils also tested include canola oil, olive oil, coconut oil and milk fat

### EXAMPLE 2

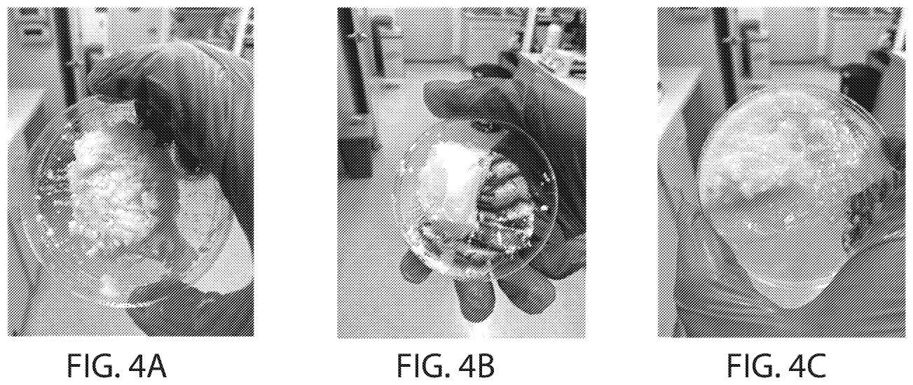

This example demonstrates the ability of fat emulsions stabilized within alginate hydrogels to mimic the consistency and texture of fat content found in native meat products. In one embodiment, a sodium alginate solution (5% w/v) was mixed with a solution of croconate oil (prepared by heating the oil to 50° C.) at a ratio of 80:20 (oil:alginate) and mixed vigorously. A calcium chloride solution (1% v/v final concentration was then added to the solution to induce gelation of the fat replica. The resulting fat was granular and brittle, but was able to hold its form; the texture resembled fatty tissue typically found in animal muscle (FIG. 4A).

In another embodiment, the ratio of oil to alginate was 50:50 and a surfactant, sorbitan 80 (0.1%) was added to the solution prior to emulsification. The addition of the surfactant improved the dispersibility of the oil phase and resulted in a softer and less granular fat replica product following gelation. The fat replica produced resembled grounded or mashed fatty tissue typically found in the belly or under the skin.

In another embodiment, a fat replica was made using alginate, sunflower oil, and sorbitan 80. The ratio of alginate to sunflower oil was 50:50 and the concentration of sorbitan in the final solution was again 0.1%. Mixing and gelation with calcium chloride produced a fat replica that was soft and agranular and able to retain its form. The fat replica formed resembled hydrogenated sunflower oil or fish fat with a bright appearance. Other oils tested include milk fat, coconut fat, canola oil, sunflower oil, olive oil

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the disclosure includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A fat replica, comprising:
a fat emulsion; and
a hydrogel comprising crosslinked non-human blood plasma.

2-92. (canceled)

93. The fat replica of claim 1, wherein the fat comprises plant-based fat.

94. The fat replica of claim 1, wherein the fat comprises saturated fat.

95. The fat replica of claim 1, wherein the fat comprises unsaturated fat.

96. The fat replica of claim 1, wherein the fat replica further comprises microcarriers.

97. The fat replica of claim 96, wherein at least some of the microcarriers comprise fibrin.

98. A cultivated meat product, comprising:
a tissue mass of at least 10 g, comprising a fat emulsion and a crosslinked non-human blood plasma, wherein the tissue mass comprises no more than 0.1 wt % saturated fat.

99. The cultivated meat product of claim 98, wherein the tissue mass comprises muscle cells.

100. The cultivated meat product of claim 98, wherein the fat emulsion comprises animal fat.

101. The cultivated meat product of claim 98, wherein the fat emulsion comprises plant-based fat.

102. The cultivated meat product of claim 98, wherein the tissue mass comprises microcarriers.

103. The cultivated meat product of claim 102, wherein the microcarriers comprise a fibrin hydrogel.

104. The cultivated meat product of claim 102, wherein the microcarriers comprise a polymer.

105. The cultivated meat product of claim 102, wherein the microcarriers comprise a protein.

106. The cultivated meat product of claim 102, wherein the microcarriers comprise a polysaccharide.

107. A method, comprising:
mixing fat, a hydrogel, and a surfactant to form an emulsion;
crosslinking the hydrogel within the emulsion; and
mixing the crosslinked hydrogel and non-human animal cells to produce a tissue mass of at least 10 g.

108. The method of claim 107, wherein the hydrogel comprises non-human blood plasma.

109. The method of claim 108, wherein the non-human blood plasma contains a fibrinogen.

110. The method of claim 108, further comprising:
withdrawing a blood sample from a non-human animal; and
processing the non-human blood sample to produce the non-human blood plasma.

111. The method of claim 110, comprising processing the non-human blood sample using apheresis.
